SEQUENCE LISTING (64 sequences)

SEQ ID NO: 1 - protein, 112 aa, synthetic construct
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIK

SEQ ID NO: 2 - protein, 119 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSS

SEQ ID NO: 3 - protein, 16 aa, synthetic construct
RSSQSLVHSTGNTYLH

SEQ ID NO: 4 - protein, 7 aa, synthetic construct
KVSNRFS

SEQ ID NO: 5 - protein, 9 aa, synthetic construct
SQSTHVPFT

SEQ ID NO: 6 - protein, 10 aa, synthetic construct
GFTFNTYAMY

SEQ ID NO: 7 - protein, 19 aa, synthetic construct
RIRSKSNNYAIYYADSVKD

SEQ ID NO: 8 - protein, 8 aa, synthetic construct
PYSDSFAY

SEQ ID NO: 9 - protein, 16 aa, synthetic construct
RSSLSLVHSTGNTYLH

SEQ ID NO: 10 - protein, 219 aa, synthetic construct
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 449 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 445 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 122 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 124 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 16 - protein, 122 aa, synthetic construct
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 18 - protein, 107 aa, synthetic construct [sequence omitted]

SEQ ID NO: 19 - protein, 107 aa, synthetic construct
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 20 - protein, 5 aa, synthetic construct
SYAFS

SEQ ID NO: 21 - protein, 16 aa, synthetic construct
RVIPFLGIANSAQKFQ

SEQ ID NO: 22 - protein, 11 aa, synthetic construct
DDIAALGPFDY

SEQ ID NO: 23 - protein, 11 aa, synthetic construct
RASQGISSWLA

SEQ ID NO: 24 - protein, 7 aa, synthetic construct
AASSLQS

SEQ ID NO: 25 - protein, 9 aa, synthetic construct
QQYNSYPRT

SEQ ID NO: 26 - protein, 5 aa, synthetic construct
SFAMS

SEQ ID NO: 27 - protein, 17 aa, synthetic construct
AISGSGGGTYYADSVKG

SEQ ID NO: 28 - protein, 13 aa, synthetic construct
DKILWFGEPVFDY

SEQ ID NO: 29 - protein, 11 aa, synthetic construct
RASQSVSSYLA

SEQ ID NO: 30 - protein, 7 aa, synthetic construct
DASNRAT

SEQ ID NO: 31 - protein, 10 aa, synthetic construct
QQRSNWPPTF

SEQ ID NO: 32 - protein, 121 aa, synthetic construct
QVQLVQSGAEVKKPGASVKVSCKASGYTFTSYSINWVRQAPGQGLEWMGYIDPNRGNTNYAQKFQGRVTMTRDTSISTAYMELSSLRSEDTAVYYCAREYIYFIHGMLDFWGQGTLVTVSS

SEQ ID NO: 33 - protein, 114 aa, synthetic construct
DIVMTQSPLSLPVTPGEPASISCRSSQSLLFIDGNNYLNWYLQKPGQSPQLLIYLGSNRASGVPDRFSGSGSGTDFTLKISRVEAEDVGVYYCQQYSSKSATFGQGTKVEIKRT

SEQ ID NO: 34 - protein, 122 aa, synthetic construct
QVQLVESGGGLVQPGGSLRLSCAASGFTFSNYGMHWVRQAPGKGLEWVSNIRSDGSWTYYADSVKGRFTISRDNSKNTLYLQMNSLRAEDTAVYYCARRYWSKSHASVTDYWGQGTLVTVSS

SEQ ID NO: 35 - protein, 109 aa, synthetic construct
DIQMTQSPSSLSASVGDRVTITCRASQDISAFLNWYQQKPGKAPKLLIYKVSNLQSGVPSRFSGSGSGTDFTLTISSLQPEDFATYYCQQAYSGSITFGQGTKVEIKRT

SEQ ID NO: 36 - protein, 122 aa, synthetic construct
QVQLVESGGGLVQPGGSLRLSCAASGFTFSSYGMHWVRQAPGKGLEWVSNIYSDGSNTFYADSVKGRFTISRDNSKNTLYLQMNSLRAEDTAVYYCARNMYRWPFHYFFDYWGQGTLVTVSS

SEQ ID NO: 37 - protein, 108 aa, synthetic construct
DIELTQPPSVSVAPGQTARISCSGDNIGNKYVSWYQQKPGQAPVVVIYGDNNRPSGIPERFSGSNSGNTATLTISGTQAEDEADYYCSSYDSSYFVFGGGTKLTVLGQ

SEQ ID NO: 38 - protein, 119 aa, synthetic construct
QVQLVESGGGLVQPGGSLRLSCAASGFTFSSNGMSWVRQAPGKGLEWVSNISYLSSSTYYADSVKGRFTISRDNSKNTLYLQMNSLRAEDTAVYYCARFYGYFNYADVWGQGTLVTVSS

SEQ ID NO: 39 - protein, 109 aa, synthetic construct
DIELTQPPSVSVAPGQTARISCSGDNIGHYYASWYQQKPGQAPVLVIYRDNDRPSGIPERFSGSNSGNTATLTISGTQAEDEADYYCQSYDYLHDFVFGGGTKLTVLGQ

SEQ ID NO: 40 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 41 - protein, 14 aa, synthetic construct
SKRNIQFSCKNIYR

SEQ ID NO: 42 - protein, 14 aa, synthetic construct
EKVQTLEAWVIHGG

SEQ ID NO: 43 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 14 aa, synthetic construct
SKRNIQFSCKNIYR

SEQ ID NO: 46 - protein, 14 aa, synthetic construct
EKVQTLEAWVIHGG

SEQ ID NO: 47 - protein, 5 aa, synthetic construct
SFAMS

SEQ ID NO: 48 - protein, 16 aa, synthetic construct
AISGSGGGTYYADSVK

SEQ ID NO: 49 - protein, 13 aa, synthetic construct
DKILWFGEPVFDY

SEQ ID NO: 50 - protein, 11 aa, synthetic construct
RASQSVSSYLA

SEQ ID NO: 51 - protein, 7 aa, synthetic construct
DASNRAT

SEQ ID NO: 52 - protein, 10 aa, synthetic construct
QQRSNWPPTF

SEQ ID NO: 53 - protein, 122 aa, synthetic construct [sequence omitted]

SEQ ID NO: 54 - protein, 107 aa, synthetic construct [sequence omitted]

SEQ ID NO: 55 - protein, 452 aa, synthetic construct; identical to SEQ ID NO: 43

SEQ ID NO: 56 - protein, 214 aa, synthetic construct; identical to SEQ ID NO: 44

SEQ ID NO: 57 - protein, 122 aa, synthetic construct; identical to SEQ ID NO: 14

SEQ ID NO: 58 - protein, 107 aa, synthetic construct; identical to SEQ ID NO: 17

SEQ ID NO: 59 - protein, 122 aa, synthetic construct; identical to SEQ ID NO: 16

SEQ ID NO: 60 - protein, 107 aa, synthetic construct [sequence omitted]

SEQ ID NO: 61 - protein, 120 aa, synthetic construct
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 62 - protein, 109 aa, synthetic construct
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 63 - protein, 120 aa, synthetic construct [sequence omitted]

SEQ ID NO: 64 - protein, 107 aa, synthetic construct [sequence omitted]

(19) United States
(12) Patent Application Publication
(10) Pub. No.: US 2025/0075007 A1
(43) Pub. Date: Mar. 6, 2025

(54) Treatment of Al Amyloidosis with the Combination of Monoclonal Antibodies Agains Immunoglobulin Light Chains and the CD38 Cell Membrane Molecule on Antibody-Producing And Other Immune Cells

(71) Applicants: Prothena Biosciences Limited, Dublin 2 (IE); Tufts Medical Center, Inc., Boston, MA (US)

(72) Inventors: Raymond COMENZO, Boston, MA (US); Wagner Marcelo ZAGO, San Carlos, CA (US); Nina Mercedes ASHTON, Brisbane, CA (US)

(21) Appl. No.: 18/920,634

(22) Filed: Oct. 18, 2024

Related U.S. Application Data
(63) Continuation of application No. 17/429,876, filed on Aug. 10, 2021.
(60) Provisional application No. 62/804,721, filed on Feb. 12, 2019.

(51) Int. Cl. C07K 16/42; A61K 39/00; A61K 45/06; A61P 7/00; A61P 35/02; C07K 16/28

(52) U.S. Cl. CPC C07K 16/42; A61K 45/06; A61P 7/00; A61P 35/02; C07K 16/2896; A61K 2039/507; C07K 2317/21; C07K 2317/24; C07K 2317/76

(57) ABSTRACT

Treatment of AL Amyloidosis with the Combination of Monoclonal Antibodies against Immunoglobulin Light Chains and Aggregates of Immunoglobulin Light Chains and the CD38 Cell Membrane Molecule on Antibody-Producing and Other Immune Cells

### CROSS-REFERENCE TO RELATED APPLICATIONS

This application is a continuation of U.S. patent application Ser. No. 17/429,876, filed on Aug. 10, 2021, which is a National Stage application under 35 U.S.C. § 371 of International Patent Application No. PCT/US2019/066648, filed on Dec. 16, 2019, which claims the benefit of U.S. Provisional Patent Application Ser. No. 62/804,721, filed on Feb. 12, 2019. The disclosures of the prior applications are incorporated by reference in their entirety.

### FIELD

The disclosure relates to the technical fields of immunology and medicine.

### SEQUENCE LISTING

This application contains a Sequence Listing that has been submitted electronically as an XML file named “50887-0020002 ST26 SL.XML.” The XML file, created on Oct. 17, 2024, is 62,501 bytes in size. The material in the XML file is hereby incorporated by reference in its entirety.

### BACKGROUND

Amyloid light-chain (AL) amyloidosis involves a hematological disorder caused by clonal plasma cells that produce immunoglobulin light chains that can misfold and contribute to disease. Overproduction of misfolded light chain by plasma cells results in deposits of abnormal AL protein (amyloid) in the tissues and organs of individuals with AL amyloidosis. Clinical features of AL amyloidosis include a constellation of symptoms and organ dysfunction that can include cardiac, renal, and hepatic dysfunction, gastrointestinal involvement, neuropathies and macroglossia. The mechanisms by which amyloidogenic immunoglobulin light chains result in organ dysfunction are not well characterized, however, it is hypothesized that both amyloid deposits and prefibrillar aggregates may contribute to cytotoxic effects on organs observed in patients with AL amyloidosis. AL amyloidosis is a disease entity of its own, although AL amyloidosis can occur concurrently in a subset of patients with multiple myeloma (up to 15%) or monoclonal gammopathy of unknown significance (MGUS; up to 9%). Patients with cardiac involvement have high-risk disease as evidenced by the fact that approximately 25% of patients with cardiac involvement die within 6 months of diagnosis despite current therapeutic advances.

AL amyloidosis is a rare disorder with an estimated incidence of 8 in 1,000,000 people. Only 1200 to 3200 new cases of AL amyloidosis are reported each year in the United States. Two thirds of patients with AL amyloidosis are male and less than 5% of patients are under 40 years of age. Both the causes and origins of AL amyloidosis remain poorly understood.

Current treatment of patients with AL amyloidosis is aimed at reducing or eliminating the bone marrow disorder, i.e. the plasma cells that are responsible for producing the light chains, thereby limiting or halting the production of amyloid. The most aggressive treatment options include stem cell transplant and high-dose chemotherapy for those patients who can tolerate it. Other treatment regimens include combinations of drugs often used to treat hematological malignancies, such as melphalan, prednisone, dexamethasone and proteasome inhibitors such as bortezomib, in an attempt to reduce light chain production. CD38 antibodies such as daratumumab (DARZALEX®) and Isatuximab have been developed for the treatment of multiple myeloma. Daratumumab attaches to CD38 present on the surface of myeloma cells. It is thought to work both by killing tumor cells directly and my stimulating an immune response against cancer cells.

There are no currently approved treatments for AL amyloidosis, and none that directly target potentially toxic forms of the amyloidogenic proteins. While some treatment options may ameliorate some of the morbidity associated with AL amyloidosis, few if any have been demonstrated to achieve high rates of hematologic and cardiac responses in patients.

Thus, there is a need for therapies that improve the outcome of patients with AL amyloidosis.

### SUMMARY

The present disclosure relates to methods of treating patients with AL amyloidosis with antibodies that target different proteins associated with AL amyloidosis or a plasma cell dyscrasia and provides a method of treating a patient with AL amyloidosis, comprising administering an effective dosage of an antibody which specifically binds to amyloid light chains and an antibody that specifically binds to CD38, for example, a chimeric or humanized monoclonal antibody to CD38. Typically, the dosage is effective to achieve an improvement in hematologic or cardiac or other organ function. The dosage can be effective to achieve an improvement in both hematologic and organ function, for example, cardiac function. In some methods, the amyloid light chain antibody or the CD38 antibody is a Fab, Fab′, F(ab′)₂, F(ab)c, Dab, nanobody or Fv.

In some of the methods disclosed herein, the amyloid light chain antibody competes for binding to human amyloid A peptide or human kappa or human lambda light chain immunoglobulin with antibody 2A4 (ATCC Accession Number 9662) or 7D8 (ATCC Accession Number PTA-9468) or binds to the same epitope as or competes for binding to human kappa or human lambda light chain immunoglobulin with 11-1F4. In some methods, the amyloid light chain antibody is a humanized version of 2A4 or 7D8. In some methods, the antibody is a humanized bispecific or multispecific version containing combinations of 11-1F4, 2A4, and/or 7D8.

In some of the methods disclosed herein, the amyloid light chain antibody comprises a light chain variable region comprising three complementarity determining regions set forth as SEQ ID NOs: 3, 4 and 5, and a heavy chain variable region comprising three complementarity determining regions set forth as SEQ ID NOs: 6, 7 and 8.

In some of the methods disclosed herein, the light chain variable region of the amyloid light chain antibody comprises the amino acid sequence set forth as SEQ ID NO: 1. In some methods the heavy chain variable region of the amyloid light chain antibody comprises the amino acid sequence set forth as SEQ ID NO: 2. For example, the light chain variable region of the amyloid light chain antibody can comprise the amino acid sequence set forth as SEQ ID NO: 1 and the heavy chain variable region of the amyloid light chain antibody can comprise the amino acid sequence set forth as SEQ ID NO: 2.

In some of the methods disclosed herein, the amyloid light chain antibody comprises a light chain comprising the amino acid sequence set forth as SEQ ID NO:10 and a heavy chain comprising the amino acid sequence set forth as SEQ ID NO: 11, 12 or 13. In some methods, the amyloid light chain antibody comprises a light chain comprising the amino acid sequence set forth as SEQ ID NO:10 and a heavy chain comprising the amino acid sequence set forth as SEQ ID NO:12. In some methods, the amyloid light chain antibody is birtamimab (also known as NEOD001).

In some of the methods disclosed herein, the amyloid light chain antibody is present in a formulation at a concentration of about 50 mg/mL, the histidine buffer is present in the formulation concentration of about 25 mM, the trehalose is present in the formulation at a concentration of about 230 mM, the polysorbate 20 is present in the formulation at a concentration of about 0.2 g/L, and the pH is about 6.5.

In some of the methods disclosed herein, the CD38 antibody comprises a heavy chain variable region comprising the amino acid sequence set forth in SEQ ID NO:14, or 15. In some methods, the CD38 antibody comprises a light chain variable region comprising the amino acid sequence set forth in SEQ ID NO:17 or 18. In some methods, the CD38 antibody comprises heavy and light chain variable region amino acid sequences as set forth in (a) SEQ ID NOs:14 and 17, respectively; (b) SEQ ID NOs:15 and 18, respectively; or (c) SEQ ID NOs:16 and 19, respectively; (d) SEQ ID NOs: 43 and 44, respectively; (e) SEQ ID NOs: 53 and 54, respectively; (f) SEQ ID NOs: 57 and 58, respectively; (g) SEQ ID NOs: 59 and 60, respectively; (h) SEQ ID NOs:61 and 62, respectively; or (i) SEQ ID NOs:63 and 64, respectively.

In some methods disclosed herein, the CD38 antibody comprises a heavy chain variable region comprising CDR1, CDR2 and CDR3 sequences comprising the amino acid sequences set forth in SEQ ID NOs:47, 48, and 49, respectively, and a light chain variable region comprising CDR1, CDR2 and CDR3 sequences comprising the amino acid sequences set forth in SEQ ID NOs:50, 51, and 52, respectively.

In some of the methods disclosed herein, the CD38 antibody comprises a heavy chain variable region comprising CDR1, CDR2 and CDR3 sequences comprising the amino acid sequences set forth in SEQ ID NOs:26, 27 and 28, respectively, and a light chain variable region comprising CDR1, CDR2 and CDR3 sequences comprising the amino acid sequences set forth in SEQ ID NOs:29, 30 and 31, respectively.

In some of the methods disclosed herein, the CD38 antibody comprises a heavy chain variable region comprising CDR1, CDR2 and CDR3 sequences from the antibody comprising the amino acid sequence set forth in SEQ ID NO:32, and a light chain variable region comprising CDR1, CDR2 and CDR3 sequences from the antibody comprising the amino acid sequences set forth in SEQ ID NO:33.

In some of the methods disclosed herein, the CD38 antibody comprises a heavy chain variable region comprising CDR1, CDR2 and CDR3 sequences from the antibody comprising the amino acid sequence set forth in SEQ ID NO:34, and a light chain variable region comprising CDR1, CDR2 and CDR3 sequences from the antibody comprising the amino acid sequences set forth in SEQ ID NO:35.

In some of the methods disclosed herein, the CD38 antibody comprises a heavy chain variable region comprising CDR1, CDR2 and CDR3 sequences from the antibody comprising the amino acid sequence set forth in SEQ ID NO:36, and a light chain variable region comprising CDR1, CDR2 and CDR3 sequences from the antibody comprising the amino acid sequences set forth in SEQ ID NO:37.

In some of the methods disclosed herein, the CD38 antibody comprises a heavy chain variable region comprising CDR1, CDR2 and CDR3 sequences from the antibody comprising the amino acid sequence set forth in SEQ ID NO:38, and a light chain variable region comprising CDR1, CDR2 and CDR3 sequences from the antibody comprising the amino acid sequences set forth in SEQ ID NO:39.

In some of the methods disclosed herein, the CD38 antibody is daratumumab. In some methods, the CD38 antibody comprises a heavy chain variable region comprising the amino acid sequence set forth as SEQ ID NO:43, and a light chain variable region comprising the amino acid sequence set forth as SEQ ID NO:44.

In some of the methods disclosed herein, the CD38 antibody is isatuximab or other CD38 antibody disclosed in WO 2016/187546 and US 2017/0008966, which are incorporated by reference herein in their entirety. In some methods, the CD38 antibody is isatuximab.

In some of the methods disclosed herein, the CD38 antibody binds at least to the region SKRNIQFSCKNIYR (SEQ ID NO:45) and to the region EKVQTLEAWVIHGG (SEQ ID NO:56). In some methods, the CD38 antibody comprises a heavy chain variable region comprising CDR1, CDR2 and CDR3 amino acid sequences of SEQ ID NOs:47, 48 and 49, respectively, and a light chain variable region comprising CDR1, CDR2 and CDR3 amino acid sequences of SEQ ID NOs:50, 51 and 52, respectively.

In some methods, the CD38 antibody comprises a heavy chain variable region comprising the amino acid sequence set forth as SEQ ID NO:53, and a light chain variable region comprising the amino acid sequence set forth as SEQ ID NO:54. In some methods, the CD38 antibody comprises a heavy chain comprising the amino acid sequence set forth as SEQ ID NO:55, and a light chain comprising the amino acid sequence set forth as SEQ ID NO:56.

In some of the methods disclosed herein, the CD38 antibody comprises the heavy chain CDR1, CDR2 and CDR3 and the light chain CDR1, CDR2 and CDR3 of (a) the variable heavy chain region of SEQ ID NO:57 and variable light chain region of SEQ ID NO:58; (b) the variable heavy chain region of SEQ ID NO:59 and variable light chain region of SEQ ID NO:60; (c) the variable heavy chain region of SEQ ID NO:61 and variable light chain region of SEQ ID NO:62; or (d) the variable heavy chain region of SEQ ID NO:63 and variable light chain region of SEQ ID NO:64. In some methods, the CD38 antibody comprises the variable heavy chain region of SEQ ID NO:57 and variable light chain region of SEQ ID NO:58. In some methods, the CD38 antibody comprises the variable heavy chain region of SEQ ID NO:59 and variable light chain region of SEQ ID NO:60. In some methods, the CD38 antibody comprises the variable heavy chain region of SEQ ID NO:61 and variable light chain region of SEQ ID NO:62. In some methods, the CD38 antibody comprises the variable heavy chain region of SEQ ID NO:63 and variable light chain region of SEQ ID NO:64.

In some methods, the antibody is a humanized bispecific or multispecific version containing combinations of daratumumab, isatuximab or other CD38 antibodies. In some methods, the antibody is a humanized bispecific or multispecific version containing combinations of daratumumab, isatuximab or other CD38 antibodies with 11-1F4, 2A4, and/or 7D8 or other human light chain amyloid antibodies.

In some of the methods disclosed herein, the patient previously received treatment with ixazomib, venetoclax, melphalan, prednisone, dexamethasone, bortezomib, carfilzomib, cyclophosphamide, thalidomide, pomalidomide, lenalidomide, doxorubicin, doxycycline, daratumumab, autologous transplant or a combination thereof. In some methods, the patient had not responded to therapy with bortezomib.

In some of the methods disclosed herein, the amyloid light chain antibody and the CD38 antibody are administered to the patient by intravenous infusions separated by two days. In some methods, the amyloid light chain antibody is administered first. Alternatively, the CD38 antibody can be administered first.

In some of the methods disclosed herein, the patient achieved greater VGPR (very good partial response) after treatment relative to a patient receiving the CD38 antibody alone. In some methods, the patient exhibits an improvement of VGPR of greater than 85% after treatment. In some methods, the improvement is at least 88%. In some methods, the patient achieved a hematologic response in a shorter time after treatment relative to a patient receiving the CD38 antibody alone. In some methods, the patient exhibits an improvement in hematologic response in less than 60 days after treatment. In some methods, the patient exhibits an improvement in less than 45 days. In some methods, the patient exhibits an improvement in 33 days or less.

In some of the methods disclosed herein, the patient achieved a cardiac response in a shorter time after treatment relative to a patient receiving the CD38 antibody alone. In some methods, the patient achieved a greater reduction in NT-proBNP after treatment relative to a patient receiving the CD38 antibody alone. In some methods, the NT-proBNP level is reduced at least 55% after treatment. In some methods, the NT-proBNP level is reduced at least 65%. In some methods, the NT-proBNP level is reduced 74% or more.

In some of the methods disclosed herein, the dosage of the amyloid light chain antibody is from about 0.5 mg/kg to about 30 mg/kg and the amyloid light chain antibody is administered intravenously or subcutaneously at a frequency of from about weekly to about quarterly. In some methods, the duration of the treatment is at least 9 months. In some methods, the duration of the treatment is at least 12 months.

In some of the methods disclosed herein, the dosage of the amyloid light chain antibody is administered intravenously following the transfer of an amount of the formulation required for the dosage from a vial to an intravenous bag containing a liquid.

In some of the methods disclosed herein, the dosage of the amyloid light chain antibody is about 24 mg/kg and the antibody is administered intravenously every 28 days. In some methods, the dosage of CD38 antibody is 16 mg/kg.

In some of the methods disclosed herein, prior to receiving treatment with either the amyloid light chain antibody or the CD38 antibody, the patient was treatment naïve.

The disclosure also provides a combination of an amyloid light chain antibody and a CD38 antibody for use in treatment of AL amyloidosis.

In some combinations for use in treatment of AL amyloidosis, the amyloid light chain antibody competes for binding to human amyloid A peptide or human kappa or human lambda light chain immunoglobulin with antibody 2A4 (ATCC Accession Number 9662) or competes for binding to human kappa or lambda light chain immunoglobulin with 11-1F4. In some combinations, the amyloid light chain antibody is a humanized version of 2A4.

In some combinations for use in treatment of AL amyloidosis, the amyloid light chain antibody comprises a light chain variable region comprising three complementarity determining regions set forth as SEQ ID NOs: 3, 4 and 5, and a heavy chain variable region comprising three complementarity determining regions set forth as SEQ ID NOs: 6, 7 and 8.

In some combinations for use in treatment of AL amyloidosis, the light chain variable region of the amyloid light chain antibody comprises the amino acid sequence set forth as SEQ ID NO: 1. In some combinations, the heavy chain variable region of the amyloid light chain antibody comprises the amino acid sequence set forth as SEQ ID NO: 2. In some combinations, the light chain variable region comprises of the amyloid light chain antibody the amino acid sequence set forth as SEQ ID NO: 1 and the heavy chain variable region of the amyloid light chain antibody comprises the amino acid sequence set forth as SEQ ID NO: 2.

In some combinations for use in treatment of AL amyloidosis, the amyloid light chain antibody comprises a light chain comprising the amino acid sequence set forth as SEQ ID NO:10 and a heavy chain comprising the amino acid sequence set forth as SEQ ID NO: 11, 12 or 13. In some combinations, the amyloid light chain antibody comprises a light chain comprising the amino acid sequence set forth as SEQ ID NO:10 and a heavy chain comprising the amino acid sequence set forth as SEQ ID NO:12. In some combinations, the amyloid light chain antibody is birtamimab.

In some combinations for use in treatment of AL amyloidosis, the CD38 antibody comprises a heavy chain variable region comprising the amino acid sequence set forth in SEQ ID NO:14, 15, 16, 43, 53, 57, 59, 61, or 63, and the CD38 antibody comprises a light chain variable region comprising the amino acid sequence set forth in SEQ ID NO:17, 18, 19, 44, 54, 58, 60, 62, or 64. In some combinations, the CD38 antibody comprises heavy and light chain variable region amino acid sequences as set forth in (a) SEQ ID NOs:14 and 17, respectively; (b) SEQ ID NOs:15 and 18, respectively; or (c) SEQ ID NOs:16 and 19, respectively; (d) SEQ ID NOs: 43 and 44, respectively; (e) SEQ ID NOs: 53 and 54, respectively; (f) SEQ ID NOs: 57 and 58, respectively; (g) SEQ ID NOs: 59 and 60, respectively; (h) SEQ ID NOs:61 and 62, respectively; or (i) SEQ ID NOs:63 and 64, respectively.

In some combinations for use in treatment of AL amyloidosis, the CD38 antibody comprises a heavy chain variable region comprising CDR1, CDR2 and CDR3 sequences comprising the amino acid sequences set forth in SEQ ID NOs:47, 48, and 49, respectively, and a light chain variable region comprising CDR1, CDR2 and CDR3 sequences comprising the amino acid sequences set forth in SEQ ID NOs:50, 51, and 52, respectively.

In some combinations for use in treatment of AL amyloidosis, the CD38 antibody comprises a heavy chain variable region comprising CDR1, CDR2 and CDR3 sequences comprising the amino acid sequences set forth in SEQ ID NOs:20, 21 and 22, respectively, and a light chain variable region comprising CDR1, CDR2 and CDR3 sequences comprising the amino acid sequences set forth in SEQ ID NOs:23, 24 and 25, respectively.

In some combinations for use in treatment of AL amyloidosis, the CD38 antibody comprises a heavy chain variable region comprising CDR1, CDR2 and CDR3 sequences comprising the amino acid sequences set forth in SEQ ID NOs:26, 27 and 28, respectively, and a light chain variable region comprising CDR1, CDR2 and CDR3 sequences comprising the amino acid sequences set forth in SEQ ID NOs:29, 30 and 31, respectively.

In some combinations for use in treatment of AL amyloidosis, the CD38 antibody comprises a heavy chain variable region comprising CDR1, CDR2 and CDR3 sequences from the antibody comprising the amino acid sequence set forth in SEQ ID NO:32, and a light chain variable region comprising CDR1, CDR2 and CDR3 sequences from the antibody comprising the amino acid sequences set forth in SEQ ID NO:33.

In some combinations for use in treatment of AL amyloidosis, the CD38 antibody comprises a heavy chain variable region comprising CDR1, CDR2 and CDR3 sequences from the antibody comprising the amino acid sequence set forth in SEQ ID NO:34, and a light chain variable region comprising CDR1, CDR2 and CDR3 sequences from the antibody comprising the amino acid sequences set forth in SEQ ID NO:35.

In some combinations for use in treatment of AL amyloidosis, the CD38 antibody comprises a heavy chain variable region comprising CDR1, CDR2 and CDR3 sequences from the antibody comprising the amino acid sequence set forth in SEQ ID NO:36, and a light chain variable region comprising CDR1, CDR2 and CDR3 sequences from the antibody comprising the amino acid sequences set forth in SEQ ID NO:37.

In some combinations for use in treatment of AL amyloidosis, the CD38 antibody comprises a heavy chain variable region comprising CDR1, CDR2 and CDR3 sequences from the antibody comprising the amino acid sequence set forth in SEQ ID NO:38, and a light chain variable region comprising CDR1, CDR2 and CDR3 sequences from the antibody comprising the amino acid sequences set forth in SEQ ID NO:39.

In some combinations for use in treatment of AL amyloidosis, the CD38 antibody is daratumumab. In some combinations, the CD38 antibody comprises a heavy chain variable region comprising the amino acid sequence set forth as SEQ ID NO:43, and a light chain variable region comprising the amino acid sequence set forth as SEQ ID NO:44.

In some combinations for use in treatment of AL amyloidosis, the CD38 antibody is isatuximab. In some combinations, the CD38 antibody comprises a heavy chain variable region comprising the amino acid sequence set forth as SEQ ID NO:53, and a light chain variable region comprising the amino acid sequence set forth as SEQ ID NO:54.

In some combinations for use in treatment of AL amyloidosis, prior to receiving treatment with either NEOD001 or daratumumab, the patient was treatment naïve.

The present disclosure also relates to methods of treating a plasma cell dyscrasia in a patient, wherein the patient is first treated with a combination therapy of an amyloid light chain antibody and a CD38 antibody prior to receiving a plasma cell therapy. In some methods, the plasma cell ssia is selected from the group consisting of monoclonal gammopathy of undetermined significance (MGUS), asymptomatic myeloma, multiple myeloma, PC leukemia, plasmacytoma. In some methods, the plasma cell dyscrasia may lead to the development of AL amyloidosis. In some methods the co-treatment with a CD38 antibody and an amyloid light chain antibody is performed prophylactically, prior to development of AL amyloidosis. In some methods, the plasma cell therapy is selected from the group consisting of ixazomib, venetoclax, melphalan, prednisone, dexamethasone, bortezomib, carfilzomib, cyclophosphamide, thalidomide, pomalidomide, lenalidomide, doxorubicin, doxycycline and a CD38 antibody. In some methods, the plasma cell therapy is bortezomib.

In some methods, the combination therapy stabilizes or improves the patient's health to decrease the level of risk for plasma cell therapy intolerance and risk of treatment-related complications, wherein the stabilization or improvement in the patient's health is measured by very good partial response (VGPR) and/or NT-proBNP levels. In some methods, the stabilization or improvement in the patient's health comprises stabilizing or improving the patient's cardiac function prior to receiving the plasma cell therapy. In some methods, the stabilization or improvement in the patient's health comprises stabilizing or improving the patient's functional status measured by Karnofsky performance status or ECOG performance status or equivalent functional assessment tool. In some methods, the stabilization or improvement in the patient's health comprises stabilizing or improving the patient's unintentional weight loss, poor endurance, weakness, slow gait, and low physical activity. In some methods, the stabilization or improvement in the patient's health comprises stabilizing or improving the patient's instrumental activities of daily living. In some methods, the patient receives the plasma cell therapy after achieving a reduction in NT-proBNP levels relative to the patient's NT-proBNP levels prior to receiving the combination therapy of an amyloid light chain antibody and a CD38 antibody. In some methods, the NT-proBNP level is reduced at least 55%. In some methods, the NT-proBNP level is reduced at least 65%. In some methods, the NT-proBNP level is reduced 74% or more.

In some methods, the combination therapy is administered for at least 9 months before the plasma cell therapy. In some methods, the combination therapy is administered for at least 12 months before the plasma cell therapy. In some methods, the patient exhibits an improvement of VGPR of greater than 85% after the combination therapy. In some methods, the improvement of VGPR is at least 88%. In some methods, the patient exhibits an improvement in hematologic response in less than 60 days after treatment with the combination therapy prior to treatment with the plasma cell therapy. In some methods, the patient exhibits an improvement in hematologic response in less than 45 days after treatment with the combination therapy prior to treatment with the plasma cell therapy. In some methods, the patient exhibits an improvement in hematologic response in 33 between 1 day and 28 days following treatment with the combination therapy prior to treatment with the plasma cell therapy, such as, for example, 7 days, 14 days, 21 days or 28 days after treatment with the combination therapy.

In some of the methods disclosed herein, the method comprises a method of improving cardiac function in an AL patient unresponsive to treatment with NEOD001, comprising adding to the patient's treatment an effective dosing regimen of a CD38 antibody.

In some of the methods, the unresponsiveness of the patient to NEOD001 treatment is determined by NT-proBNP levels in the patient during a period following NEOD001 treatment greater than or equal to the NT-proBNP levels in the patient prior to NEOD001 treatment.

In some of the methods, the NT-pro-BNP levels are greater than the NT-proBNP levels prior to NEOD001 treatment.

In some of the methods, the period following NEOD001 treatment is at least two months.

In some of the methods, the patient has received at least two doses of NEOD001 before receiving the CD38 antibody.

In some of the methods, the patient has received at least three doses of NEOD001 before receiving the CD38 antibody.

In some of the methods, the CD38 antibody is administered after an increase of more than about 6,000 pg/mL NT-proBNP in the patient.

In some of the methods, the CD38 antibody is administered after an increase of more than about 12,000 pg/mL NT-proBNP in the patient.

In some of the methods, the CD38 antibody is administered after the levels of NT-proBNP levels increase at least about 100%. In some of the methods, the CD38 antibody is administered after the levels of NT-proBNP levels increase at least about 200%. In some of the methods, the CD38 antibody is administered after the levels of NT-proBNP levels increase at least about 300%.

In some of the methods, the AL patient has been previously been receiving NEOD001 and CyBorD.

In some of the methods, the CD38 antibody is daratumumab or isatuximab. In a method, the CD38 antibody is daratumumab.

In some of the methods, daratumumab is administered to the patient at 16 mg/kg every 28 days.

In some of the methods, NEOD001 is administered to the patient at 24 mg/kg every 28 days.

In some of the methods, the duration of treatment with the CD38 antibody is effective to reduce the patient's NT-proBNP levels at least to the levels prior to receiving NEOD001 treatment. In some of the methods, the duration is effective to reduce the patient's NT-proBNP levels below the levels prior to receiving NEOD001 treatment.

In some of the methods, the treatment includes at least one dose of the CD38 antibody. In some of the methods, the treatment include at least two doses of the CD38 antibody. In some of the methods, the treatment includes at least three doses of the CD38 antibody. In some of the methods, the duration is at least nine months. In some of the methods, the duration of the treatment is at least twelve months.

### DESCRIPTION OF THE FIGURES

FIG. 1 shows cardiac response to an example of a dual antibody therapy according to the disclosure that includes NEOD001 and daratumumab.

FIGS. 2A and 2B shows the overlap of the two curves based on the NT-proBNP response (FIG. 2A) and gradual lambda light-chain (FIG. 2B) following an example of a dual antibody therapy according to the disclosure that includes NEOD001 and daratumumab.

### DESCRIPTION

The disclosure provides methods of treating patients with AL amyloidosis, comprising administering to such patients an antibody which specifically binds to amyloid light chain in combination with an antibody that specifically binds to CD38.

### I. Definitions

The term “antibody” includes intact antibodies and antigen-binding fragments thereof. Typically, fragments compete with the intact antibody from which they were derived for specific binding to the target including separate heavy chains, light chains Fab, Fab′, F(ab′)₂, F(ab)c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. The term “amyloid light chain antibody” includes antibodies that specifically bind to a neoepitope exposed in misfolded light chains and is discussed in greater detail below. The term “CD38 antibody” includes antibodies that bind the CD38 antigen expressed on plasma cells and other lymphoid immune cells and is discussed in greater detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The phrase “substantially from a human immunoglobulin or antibody” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

### II. Methods of Treatment and Amenable Subjects

Provided herein are methods of treating a human patient showing symptoms of or diagnosed with AL amyloidosis with cardiac dysfunction, comprising administering to the patient a regime of any of the amyloid light chain antibodies described herein in combination with any of the CD38 antibodies described herein, effective to achieve positive hematologic and/or cardiac responses in the patients. Some such patients may have other systemic organ dysfunction attributed to AL amyloidosis, including dysfunction of the kidney, liver, peripheral nervous system, gastrointestinal system, autonomic nervous system, lung, and/or soft tissue or lymphatic system.

Patients amenable to treatment also include those AL amyloidosis patients who have received, are currently receiving, or will later receive an alternate therapy for treatment of AL amyloidosis or an associated condition, such as, inflammatory diseases, chronic microbial infections, malignant neoplasms, inherited inflammatory diseases, and lymphoproliferative disorders. For example, patients may also receive or have received one or more of the therapeutic agents identified herein with respect to combination therapies. As an example, patients suffering from AL amyloidosis may also receive or have received or may later receive bortezomib, ixazomib, venetoclax, melphalan, thalidomide, lenalidomide, prednisone, dexamethasone, cyclophosphamide, pomalidomide, carfilzomib, doxorubicin, doxycycline, autologous transplant or combinations thereof. For those patients who have previously received alternate therapies for the treatment of amyloid disease, such therapies may or may not have been successful by the relevant clinical measures, and likely did not improve health status. Additional examples of such prior therapies include (1) daratumumab alone, (2) CyBorD, which is a combination therapy comprising cyclophosphamide, bortezomib and dexamethasone, (3) BMDex, which is a combination of bortezomib, melphalan and dexamethasone, (4) MDex, which is a combination of melphalan and dexamethasone, (5) LDex, which is a combination of lenalidomide and dexamethasone, (6) CLD, which is a combination of cyclophosphamide, lenalidomide and dexamethasone, (7) PomDex, which is a combination of pomalidomide and dexamethasone, (8) CRd, which is a combination of lenalidomide, cyclophosphamide and dexamethasone, and (9) isatuximab. Such patients may, or may not, have experienced cardiac and/or renal improvement as a result of such treatment.

An improvement in hematologic response can be established by observing a greater than VGPR (very good partial response). One or more of the following must be present for a conclusion of VGPR: (i) serum and/or urine M-protein detectable by immunofixation but not electrophoresis; and (ii) ≥90% reduction in serum M-protein and/or urine M-protein level<100 mg/24 hours. If these are not measurable, then a ≥90% decrease in the difference between involved and uninvolved free light chain levels, provided that the serum free light chain assay shows involved level>10 mg/dL and the serum free light chain ratio is abnormal). A patient treated with the combination therapy disclosed herein can exhibit an improvement in VGPR greater than 80%, for example, at least 85%, 88% or more than 88%. The patient may achieve the greatest improvement in hematologic response in less than 75 days, for example, in less than 60 days, less than 45 days, 33 days, or less than 33 days.

An improvement in cardiac response can be established by a reduction in NT-proBNP (N-terminal pro b-type natriuretic peptide) levels (Bay et al., 2003, NT-proBNP: a new diagnostic screening tool to differentiate between patients with normal and reduced left ventricular systolic function, Heart, v. 89 (2): p150-154), and/or a reduction in the NYHA (New York Heart Association) functional classification of heart failure (Nomenclature and Criteria for Diagnosis of Diseases of the Heart and Great Vessels. 9th ed. Boston, Mass: Little, Brown & Co; 1994:253-256). A patient treated with the combination therapy disclosed herein can exhibit a reduction in NT-proBNP of greater than 50% relative to baseline, for example, greater than 55%, greater than 65%, 74% or greater than 74%.

Suitable antibodies, formulations and treatment regimens for the methods and uses disclosed herein are discussed in greater detail below.

### III. Antibodies

The methods of the disclosure include administering to an AL amyloidosis patient an amyloid light chain antibody and a CD38 antibody.

An amyloid light chain antibody is an antibody that specifically binds to immunoglobulin light chain. Examples include antibodies that compete with 11-1F4 (also known as CAEL-101) for binding to immunoglobulin light chain and antibodies that compete with 2A4 (ATCC Accession Number 9662) or 7D8 (ATCC Accession Number PTA-9468) for binding to human amyloid A peptide or human kappa or human lambda light chain immunoglobulin, or specifically bind to the same epitope as or compete for binding to human kappa or human lambda light chain immunoglobulin with 11-1F4 (U.S. Pat. No. 8,105,594), 2A4 or 7D8 (U.S. Pat. No. 7,928,203). In some methods, the antibody is a humanized version of 2A4. In some methods, the antibody comprises a light chain variable region comprising three complementarity determining regions set forth as SEQ ID NOs: 3, 4 and 5, and a heavy chain variable region comprising three complementarity determining regions set forth as SEQ ID NOs: 6, 7 and 8. In some methods, the light chain variable region comprises the amino acid sequence set forth as SEQ ID NO: 1. In some methods, the heavy chain variable region comprises the amino acid sequence set forth as SEQ ID NO: 2. In some methods, the light chain variable region comprises the amino acid sequence set forth as SEQ ID NO: 1 and the heavy chain variable region comprises the amino acid sequence set forth as SEQ ID NO: 2. In some methods, the antibody comprises a light chain variable region comprising three complementarity determining regions set forth as SEQ ID NOs: 9, 4 and 5, and a heavy chain variable region comprising three complementarity determining regions set forth as SEQ ID NOs: 6, 7 and 8.

In other methods, the antibody comprises light chain and heavy chain variable regions of a murine, chimeric, or humanized 2A4 antibody, or of a murine, chimeric, or humanized 7D8antibody, as described in U.S. Pat. No. 7,928,203 and PCT International Publication No. WO 2009/086539, each of which is incorporated herein by reference in its entirety, and the light chain and heavy chain variable region sequences described in the referenced patent and publication are specifically incorporated by reference herein. Some formulations for the methods disclosed herein are described in U.S. Pat. No. 9,089,529 and PCT International Publication No. WO 2013/063284.

In some methods, the antibody comprises a light chain comprising an amino acid sequence set forth as SEQ ID NO: 10 and a heavy chain comprising an amino acid sequence set forth as any one of SEQ ID NOs: 11-13. For example, the antibody can comprise a light chain comprising an amino acid sequence set forth as SEQ ID NO:10 and a heavy chain comprising an amino acid sequence set forth as SEQ ID NO:12. The antibody can include, or not include, the leader sequences of the above-noted light chain and heavy chain amino acid sequences. In some methods, the antibody is birtamimab (CAS Registry No. 1608108-91-3), also known as NEOD001.

In other methods, the antibody is a fragment of a 2A4 or 7D8 antibody, including chimeric and humanized versions thereof, such as a Fab fragment, a Fab′ fragment, a F(ab′)₂ fragment, F(ab)c, Dab, nanobody or Fv.

A CD38 antibody is an antibody that specifically binds to an epitope of CD38 on antibody-producing plasma cells and B-cells and on other lymphoid immune cells (SEQ ID NO:40). Some such antibodies bind within amino acids 44 to 206 of CD38 (SEQ ID NO:40), for example, within amino acids 44-66, 82-94, 142-154, 148-164, 158-170 or 192-206. Some CD38 antibodies bind to the region SKRNIQFSCKNIYR (SEQ ID NO:41) and the region EKVQTLEAWVIHGG (SEQ ID NO:42) Some such CD38 antibodies mediate complement dependent cytotoxicity. antibody dependent cellular cytotoxicity, antibody-dependent phagocytic activity and trogocytosis of a CD38+ target cell. In some methods, the CD38 antibody is daratumumab (CAS Registry Number 945721-28-8). Some exemplary CD38 antibodies are disclosed in U.S. Pat. No. 7,829,673 (the '673 patent), U.S. Pat. No. 8,263,746 (the '746 patent) and U.S. Pat. No. 9,249,226, which are incorporated by reference herein in their entirety.

Some CD38 antibodies comprise a heavy chain variable region comprising the CDR1, CDR2 and CDR3 sequences comprising the amino acid sequences set forth in SEQ ID NOs:20, 21 and 22, respectively (SEQ ID NOs:8, 9 and 10, respectively of the '673 patent), and a light chain variable region comprising CDR1, CDR2 and CDR3 sequences comprising the amino acid sequences set forth in SEQ ID NOs:23, 24 and 25, respectively (SEQ ID NOs:3, 4 and 5, respectively of the '673 patent). Some CD38 antibodies comprise a heavy chain variable region comprising the CDR1, CDR2 and CDR3 sequences comprising the amino acid sequences set forth in SEQ ID NOs:26, 27 and 28, respectively (SEQ ID NOs:18, 19 and 20, respectively of the '673 patent), and a light chain variable region comprising CDR1, CDR2 and CDR3 sequences comprising the amino acid sequences set forth in SEQ ID NOs:29, 30 and 31, respectively (SEQ ID NOs:13, 14 and 15, respectively of the '673 patent).

Some CD38 antibodies comprise a heavy chain variable region comprising the CDR1, CDR2 and CDR3 sequences from the antibody comprising the amino acid sequence set forth in SEQ ID NO:32 (SEQ ID NO:5 of the '746 patent), and a light chain variable region comprising CDR1, CDR2 and CDR3 sequences from the antibody comprising the amino acid sequence set forth in SEQ ID NO:33 (SEQ ID NO:13 of the '746 patent). Some CD38 antibodies comprise a heavy chain variable region comprising the CDR1, CDR2 and CDR3 sequences from the antibody comprising the amino acid sequence set forth in SEQ ID NO:34 (SEQ ID NO:6 of the '746 patent), and a light chain variable region comprising CDR1, CDR2 and CDR3 sequences from the antibody comprising the amino acid sequence set forth in SEQ ID NO:35 (SEQ ID NO:14 of the '746 patent). Some CD38 antibodies comprise a heavy chain variable region comprising the CDR1, CDR2 and CDR3 sequences from the antibody comprising the amino acid sequence set forth in SEQ ID NO:36 (SEQ ID NO:7 of the '746 patent), and a light chain variable region comprising CDR1, CDR2 and CDR3 sequences from the antibody comprising the amino acid sequence set forth in SEQ ID NO:37 (SEQ ID NO:15 of the '746 patent). Some CD38 antibodies comprise a heavy chain variable region comprising the CDR1, CDR2 and CDR3 sequences from the antibody comprising the amino acid sequence set forth in SEQ ID NO:38 (SEQ ID NO:8 of the '746 patent), and a light chain variable region comprising CDR1, CDR2 and CDR3 sequences from the antibody comprising the amino acid sequence set forth in SEQ ID NO:39 (SEQ ID NO:16 of the '746 patent).

For example, a CD38 antibody can include a heavy chain variable region comprising the amino acid sequence set forth in SEQ ID NO:14, 15 or 16 (SEQ ID NO:7, 17, 27 of the '673 patent, respectively). A CD38 antibody can include a light chain variable region comprising the amino acid sequence set forth in SEQ ID NO:17, 18 or 19 (SEQ ID NO:2, 12 or 22 of the '673 patent, respectively). Suitable CD38 antibodies can comprise combinations of the heavy chain variable regions and light chain variable regions disclosed herein. For example, some such CD38 antibodies have heavy and light chain variable region amino acid sequences as set forth in (a) SEQ ID NOs:14 and 17, respectively; (b) SEQ ID NOs:15 and 18, respectively; or SEQ ID NOs:16 and 19, respectively.

Alternatively, the CD38 antibody can be isatuximab or a CD38 antibody disclosed in WO 2016/187546 or US 2017/0008966, the publication of U.S. patent application Ser. No. 15/160,476 (the '476 application). Some CD38 antibodies bind at least to the region SKRNIQFSCKNIYR (SEQ ID NO:45) and to the region EKVQTLEAWVIHGG (SEQ ID NO:56). For example, the CD38 antibody can comprise a heavy chain variable region comprising CDR1, CDR2 and CDR3 amino acid sequences of SEQ ID NOs:47, 48 and 49, respectively, and a light chain variable region comprising CDR1, CDR2 and CDR3 amino acid sequences of SEQ ID NOs:50, 51 and 52, respectively. Some suitable CD38 antibodies comprise a heavy chain variable region comprising the amino acid sequence set forth as SEQ ID NO:53, and a light chain variable region comprising the amino acid sequence set forth as SEQ ID NO:54. In some methods, the CD38 antibody comprises a heavy chain comprising the amino acid sequence set forth as SEQ ID NO:55, and a light chain comprising the amino acid sequence set forth as SEQ ID NO:56.

Some CD38 antibodies comprise the heavy chain CDR1, CDR2 and CDR3 and the light chain CDR1, CDR2 and CDR3 of (a) the variable heavy chain region of SEQ ID NO:57 and variable light chain region of SEQ ID NO:58; (b) the variable heavy chain region of SEQ ID NO:59 and variable light chain region of SEQ ID NO:60; (c) the variable heavy chain region of SEQ ID NO:61 and variable light chain region of SEQ ID NO:62; or (d) the variable heavy chain region of SEQ ID NO:63 and variable light chain region of SEQ ID NO:64. For example, the CD38 antibody can comprise the variable heavy chain region of SEQ ID NO:57 and variable light chain region of SEQ ID NO:58. In some methods, the CD38 antibody comprises the variable heavy chain region of SEQ ID NO:59 and variable light chain region of SEQ ID NO:60. As another example, the CD38 antibody can comprise the variable heavy chain region of SEQ ID NO:61 and variable light chain region of SEQ ID NO:62. As yet another example, the CD38 antibody can comprise the variable heavy chain region of SEQ ID NO:63 and variable light chain region of SEQ ID NO:64.

In some methods, the patient is administered birtamimab and daratumumab. In other methods, the patient is administered birtamimab and isatuximab. As discussed in greater detail below, the antibodies can be administered as a pharmaceutical formulation.

### IV. Pharmaceutical Formulations and Products

In some methods disclosed herein, the antibody can be administered to an AL amyloidosis patient as a pharmaceutical formulation, for example, comprising in addition to the antibody, a histidine buffer, trehalose, and polysorbate 20, such as the formulations disclosed in U.S. Pat. No. 9,884,020, which is hereby incorporated by reference in its entirety.

In some methods, the amyloid light chain antibody and the CD38 antibody are formulated together. In other methods, the amyloid light chain antibody and the CD38 antibody are prepared in different pharmaceutical formulations. In some such methods, the amyloid light chain antibody is prepared in any of the formulations described above and the CD38 antibody is prepared in a different formulation, such as, for example, any of the formulations disclosed in US patent publication number US2017/0121414 or U.S. Pat. No. 9,364,542, which are hereby incorporated by reference in their entirety.

### V. Treatment Regimes

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

Desired outcomes of the treatments disclosed herein vary according to the amyloid disease and patient profile and are readily determinable to those skilled in the art. Desired outcomes include an improvement in the patient's health status. Generally, desired outcomes include measurable indices such as reduction or clearance of pathologic amyloid fibrils, decreased or inhibited amyloid aggregation and/or deposition of amyloid fibrils, and increased immune response to pathologic and/or aggregated amyloid fibrils. Desired outcomes also include amelioration of amyloid disease-specific symptoms. For example, desired outcomes for the treatment of AL amyloidosis include a decrease in the incidence or severity of known symptoms, including organ dysfunction, peripheral and autonomic neuropathy, carpal tunnel syndrome, macroglossia, restrictive cardiomyopathy, arthropathy of large joints, immune dyscrasias, myelomas, as well as occult dyscrasias. Desired outcomes of the disclosed therapies are generally quantifiable measures as compared to a control or baseline measurement. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed antibody formulations. In this case, efficacy of the disclosed antibody formulations is assessed by a shift or trend away from measurable indices in the untreated control. Alternatively, a control individual is a healthy individual, who is about the same age as the individual being treated. In this case, efficacy of the disclosed antibody formulations is assessed by a shift or trend toward from measurable indices in the healthy control. Changes or improvements in response to therapy are generally statistically significant and described by a p-value less than or equal to 0.1, less than 0.05, less than 0.01, less than 0.005, or less than 0.001 may be regarded as significant.

Treatment typically entails multiple dosages over a period of time. Treatment can be monitored by assaying antibody, or employing radiolabeled SAP Scintigraphy over time. If the response falls, a booster dosage may be indicated. In addition, the response of patients with AL amyloidosis to treatment can be monitored by assessing cardiac markers, such as NT-proBNP and/or troponin, serum creatine, and/or alkaline phosphatase; by performing serum free light chain (SFLC) assays, quantitative immunoglobulin assays, biopsies, serum protein electrophoresis (SPEP), urine protein electrophoresis (UPEP), serum, urine immunofixation electrophoresis (IFE), and/or organ imaging techniques. An exemplary complete response (CR) can be determined from response criteria including negative IFE of serum and urine, normal kappa/lamda (κ/λ) ratio and/or <5% plasma cells in bone marrow. An exemplary very good partial response (VGPR) can be determined from a dFLC of <40 mg/L. An exemplary partial response (PR) can be determined from a dFLC decrease of ≥50%. In the kidney, a response to treatment can be determined, for example, from a >50% reduction (e.g., >0.5g/24 hours) in 24 hour urine protein excretion in the absence of either a reduction in eGFR of >25% or an increase in serum creatine of ≥0.5 mg/dL. In the liver, a response to treatment can be determined, for example, from a ≥50% reduction in initially elevated alkaline phosphatase or a ≥2 cm reduction in liver size on CT scan or MRI. In the heart, a response to treatment can be determined, for example, from a >30% and >300 ng/L reduction in NT-proBNP in patients with baseline of NT-proBNP of >650 ng/L. In the kidney, a response to treatment can be determined, for example, from a >30% decrease in proteinuria or a decrease in proteinuria to <0.5 g/24 hours in the absence of renal progression. Neuropathy responders are generally characterized by <2 point increase in NIS-LL from baseline. Improvement in neuropathy (e.g., improved nerve function) is determined from a decrease in the NIS-LL from baseline.

The antibody formulation can be administered intravenously or subcutaneously in dosage ranges from about 0.5 mg/kg to about 30 mg/kg of the body weight. For example, dosages can be about 0.5 mg/kg body weight, about 1.0 mg/kg, about 1.5 mg/kg, about 2.0 mg/kg, about 4.0 mg/kg, about 5.0 mg/kg, about 8.0 mg/kg, about 10 mg/kg, about 15 mg/kg, about 16 mg/kg, about 20 mg/kg, about 24 mg/kg, about 25 mg/kg, or about 30 mg/kg body weight. For intravenous dosing, an amount of the antibody formulation sufficient to achieve the desired dosage for the individual patient is transferred from one or more vials to one or more intravenous bags containing a liquid (e.g., saline) and administered to the patient. In some methods, a dose of about 24 mg/kg of any of the amyloid light chain antibodies disclosed herein, such as, for example, birtamimab is administered to the patient. In some methods, a dose of about 16 mg/kg of any of the CD38 antibodies disclosed herein, such as, for example, daratumumab is administered to the patient.

Antibody is usually administered on multiple occasions. An exemplary treatment regime entails administration once per every two weeks, once a month, or once every 3 to 6 months. For example, patients can receive the antibody formulation once every four weeks as a cycle, for example every twenty-eight days. The dosing frequency can be adjusted depending on the pharmacokinetic profile of the antibody formulation in the patient. For example, the half-life of the antibody may warrant a frequency of dosing every two weeks.

In some methods, the pharmaceutical formulation is administered intravenously every 28 days with an amyloid light chain antibody dosage of about 24 mg/kg. For example, some patients may receive an intravenous dose of about 24 mg/kg any of the amyloid light chain antibodies disclosed herein, such as, for example, birtamimab, every 28 days. Some such patients receive an intravenous dose of any of the CD38 antibodies disclosed herein, such as, for example, daratumumab at a frequency every 28 days, for example at a dose of 16 mg/kg. Some patients receive the CD38 antibody weekly. Some patients receive the CD38 antibody every two weeks. Some patients receive the CD38 antibody more frequently initially, and the less frequently over time. For example, a patient may receive the CD38 antibody weekly for a period of time, followed by every two weeks for a period of time, followed by monthly or every 28 days thereafter for the duration of treatment. One such dosing regimen is weekly doses of a CD38 antibody such as daratumumab for eight weeks, followed by dosing every two weeks for four months, followed by monthly dosing thereafter for the duration of treatment.

For some such patients, the amyloid light chain antibody formulation transferred to the intravenous bag was first reconstituted from a lyophilized formulation to a formulation having a pH of about 6.5 and comprising about 50 mg/ml amyloid light chain antibody such as birtamimab, about 25 mM histidine buffer, about 230 mM trehalose and about 0.2 g/L polysorbate 20.

For some patients the desired dosage of one or more of the amyloid light chain antibody and/or the CD38 antibody can be administered subcutaneously without dilution from a vial containing any of the formulations disclosed herein.

In some methods disclosed herein, the antibody is administered to the patient for at least 9 months, at least 12 months, or for a longer period of time.

When performing the combination therapy with amyloid light chain antibody and the CD38 antibody, the two antibodies can be administered simultaneously or sequentially in any order, i.e., one antibody is administered prior to administering the other antibody, concurrently with the other antibody, or subsequent to administration of the other antibody. For example, a combination therapy may be performed by administering the first antibody prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) administering the second antibody. In some methods, the amyloid light chain antibody and CD38 antibody are administered to the patient on the same day, for example, simultaneously or sequentially in one day. In some methods, the two antibodies are administered separately at least 24 hours apart, 2 days apart, 3 days apart, 4 days apart 5 days apart, 6 days apart or a week apart. Where the two antibodies are not being administered simultaneously, in some methods the CD38 antibody is administered first, followed by the amyloid light chain antibody. In other methods the amyloid light chain antibody is administered first followed by the CD38 antibody.

The dosage, frequency and mode of administration of each component of the combination can be controlled independently. For example, one therapeutic agent/therapy may be administered orally three times per day, while the second therapeutic agent/therapy may be administered intramuscularly once per day. Combination therapy may be given in on-and-off cycles that include rest periods. The compounds may also be admixed or otherwise formulated together such that one administration delivers both compounds. In this case, each therapeutic agent is generally present in an amount of 1-95% by weight of the total weight of the composition. Alternatively, an antibody formulation disclosed herein and a second therapeutic agent can be formulated separately and in individual dosage amounts. Drug combinations for treatment can be provided as components of a pharmaceutical pack.

In some combinations for use in treatment of AL amyloidosis, prior to receiving treatment with either NEOD001 or daratumumab, the patient was treatment naïve. For example, the patient has previously received any treatment for AL amyloidosis, even standard of care treatment.

In some of the methods as disclosed herein, the patient is treated with either the amyloid light chain antibody or the CD38 antibody prior to treatment for a plasma cell dyscrasia. Plasma cell dyscrasias (PCD; also termed plasma cell disorders or plasma cell proliferative diseases) are a spectrum of progressively more severe monoclonal gammopathies in which a clone or multiple clones of plasma cells over-produce and secrete into the blood stream an immunoglobulin or fragment thereof. PCDs can include, but are not limited to monoclonal gammopathy of undetermined significance (MGUS), asymptomatic myeloma, multiple myeloma, PC leukemia, plasmacytoma. In some such methods, patients are first treated with the amyloid light chain antibody and the CD38 antibody to stabilize or improve the patient's health (for example, the patient's cardiac function), prior to treatment with a plasma cell therapy such as one or more of ixazomib, venetoclax, melphalan, prednisone, dexamethasone, bortezomib, carfilzomib, cyclophosphamide, thalidomide, pomalidomide, lenalidomide, doxorubicin, doxycycline or CD38 antibody. The treatment for a plasma cell therapy may include a CD38 antibody, assuming the treatment for the plasma cell therapy occurs after the combination treatment with the amyloid light chain antibody and the CD38 antibody. In one such method of the disclosure, the treatment with combination of the amyloid light chain antibody and the CD38 antibody is followed by the treatment for the plasma cell dyscrasia wherein the treatment for the dyscrasia is a CD38 antibody or wherein the treatment for the dyscrasia is bortezomib.

The pretreatment with the amyloid light chain antibody and the CD38 antibody enhances may enhance the ability of the patient to tolerate the side effects of the subsequent plasma cell therapy. The pretreatment may also decrease the level of risk for plasma cell therapy intolerance and risk of treatment-related complications. In some methods the combination treatment with a CD38 antibody and an amyloid light chain antibody is performed prophylactically, prior to development of AL amyloidosis.

In such methods, the order of administration and dosing regimen of the amyloid light chain antibody and the CD38 antibody can be as described herein. For example, a patient may be treated with the combination of the amyloid light chain antibody and the CD38 antibody to an improvement in patient health as described herein. For instance, the improvement may be a reduction in NT-proBNP prior to administration of a plasma cell therapy, such as, for example, bortezomib. For instance, a patient treated with the combination therapy disclosed herein can exhibit a reduction in NT-proBNP of greater than 50% relative to baseline, for example, greater than 55%, greater than 65%, 74% or greater than 74%, prior to treatment with a plasma cell therapy. Other measures of improvements of the patient's health are described herein, e.g., by measuring other cardiac markers (troponin, serum creatine, and/or alkaline phosphatase and by performing serum free light chain (SFLC) assays, quantitative immunoglobulin assays, biopsies, serum protein electrophoresis (SPEP), urine protein electrophoresis (UPEP), serum, urine immunofixation electrophoresis (IFE), and/or organ imaging techniques. Other measure of improvement also include stabilization or improvement the patient's functional status measured by Karnofsky performance status or ECOG performance status or equivalent functional assessment tool, improvement of the patient's unintentional weight loss, poor endurance, weakness, slow gait, and low physical activity and/or improvement of patient's instrumental activities of daily living.

Accordingly, some of the methods as disclosed herein relate to methods of treating a plasma cell dyscrasia in a patient, wherein the patient is first treated with a combination therapy of an amyloid light chain antibody and a CD38 antibody prior to receiving a plasma cell therapy. The various manifestations of PCD can require different treatment regiments. PCD therapies can involve the use of hematopoietic stem cell transplants (HSCT), and/or chemotherapeutic agents. In some methods, the plasma cell therapy is one or more of ixazomib, venetoclax, melphalan, prednisone, dexamethasone, bortezomib, carfilzomib, cyclophosphamide, thalidomide, pomalidomide, lenalidomide, doxorubicin and doxycycline. In some methods, the plasma cell therapy is bortezomib.

In some such methods, the patients are first treated with a combination therapy of the amyloid light chain antibody and the CD38 antibody to stabilize or improve the patient's health (for example, the patient's cardiac function), prior to treatment with the plasma cell therapy. An improvement in a patient's heath can be determined, for example, by determining a reduction in NT-proBNP of greater than 50% relative to baseline in the patient. In particular, improvement in a patient's health many be exhibited by a reduction in NT-proBNP greater than 55%, greater than 65%, 74% or greater than 74%, relative to baseline. The improvement in the patient's health can enhance the ability of the patient to tolerate the side effects of the subsequent plasma cell therapy. In such methods, the order of administration and dosing regimen of the combination therapy of the amyloid light chain antibody and the CD38 antibody can be as described herein. For example, a patient may be treated with the combination of the amyloid light chain antibody and the CD38 antibody to achieve a reduction in NT-proBNP of at least 55% prior to administration of a plasma cell therapy, such as, for example, bortezomib.

The combination antibody therapy as described herein may be overlap with the plasma cell therapy in order to maintain the patient's improvement in health that was obtained prior to the plasma cell therapy. Alternatively, the combination antibody therapy may be stopped immediately before, days before, weeks or months before the plasma cell therapy as long at the patient's health has improved to the extent the patient is can more readily tolerate the side effects of the plasma cell therapy. For example, the combination therapy may be administered for at least 9 months or for at least 12 months using a dosing regimen as described herein before the plasma cell therapy and terminated prior to or during the antibody combination therapy.

In some methods, the patient exhibits an improvement of VGPR of greater than 85% after the combination therapy. In some methods, the improvement of VGPR is at least 88%. In some methods, the patient exhibits an improvement in hematologic response in less than 60 days after treatment with the combination therapy prior to treatment with the plasma cell therapy. In some methods, the patient exhibits an improvement in hematologic response in less than 45 days after treatment with the combination therapy prior to treatment with the plasma cell therapy. In some methods, the patient exhibits an improvement in hematologic between 1 day and 28 days following treatment with the combination therapy prior to treatment with the plasma cell therapy, such as, for example, 7 days, 14 days, 21 days or 28 days after treatment with the combination therapy.

The amyloid light chain antibody may be as described herein and as provided in the sequences for the amyloid light chain antibodies. Similarly, the CD38 antibody is as described herein and may be, for example, daratumumab. Similar, as described herein, the dosages for the amyloid light chain antibody may from about 0.5 mg/kg to about 30 mg/kg, which may be administered intravenously or subcutaneously at a frequency of from about weekly to about quarterly. In one method herein, the dosage of the amyloid light chain is about 24 mg/kg and the antibody is administered intravenously every 28 days and may include the formulations described herein.

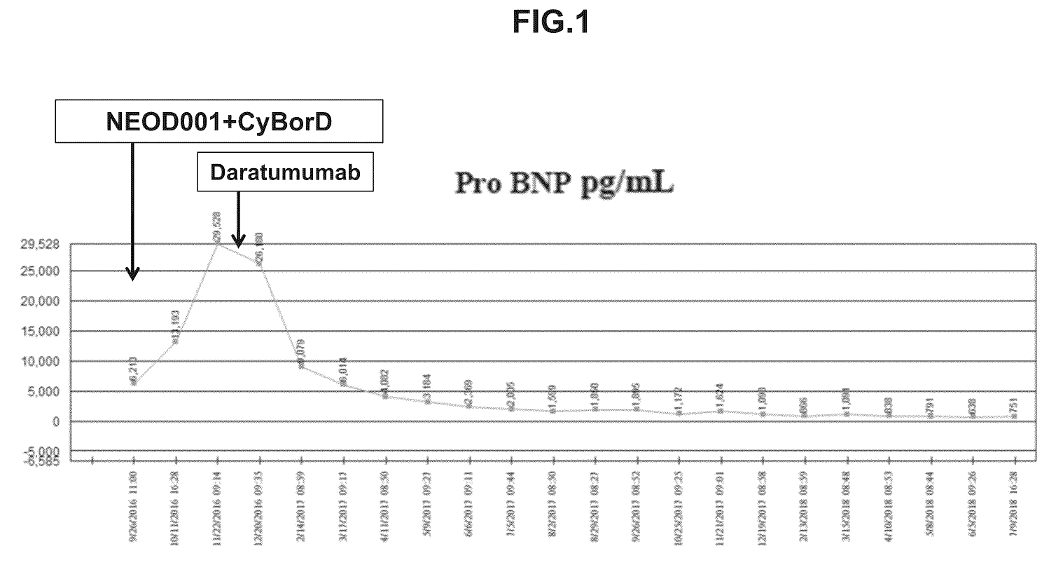

In some of the methods disclosed herein, the method comprises a method of improving cardiac function in an AL patient unresponsive to treatment with NEOD001, comprising adding to the patient's treatment an effective dosing regimen of a CD38 antibody. Patients unresponsive to NEOD001 include patients that are treated with CyBorD (cyclophosphamide, bortezomib, dexamethasone). Patient response may be measured as a cardiac response, such as NT-proBNP. Non-responsive patients includes those with no improvement to NEOD001 (with or without CyBorD) or patients whose conditions continues to worsen as shown in FIG. 1. For instance, the unresponsiveness of the patient to NEOD001 treatment may be determined by NT-proBNP levels in the patient during a period following NEOD001 treatment greater than or equal to the NT-proBNP levels in the patient prior to NEOD001 treatment. For example, the NT-pro-BNP levels are greater than the NT-proBNP levels prior to NEOD001 treatment.

Dosing regimens can vary and may include the period following NEOD001 treatment of at least two months before the administration of the CD38 antibody. In some instance, the patient may have received at least two doses or three doses of NEOD001 before receiving the CD38 antibody. The CD38 antibody is administered after an increase of more than about 2,000 about 15,000 pg/mL NT-pro-BNP. For instance the increase in NT-proBNP may be 2,000, 3,000, 4,000, 5,000, 6,000, 7,000, 8,000, 9,000, 10,000, 11,000, 12,000, 13,000, 14,000 or 15,000 pg/mL NT-proBNP in the patient. In some of the methods, the CD38 antibody is administered after an increase of more than about 12,000 pg/mL NT-proBNP in the patient.

In some of the methods of the disclosure, the CD38 antibody is administered after the levels of NT-proBNP levels increase at least about 100%. In some of the methods, the CD38 antibody is administered after the levels of NT-proBNP levels increase at least about 200%. In some of the methods, the CD38 antibody is administered after the levels of NT-proBNP levels increase at least about 300%. As further described herein, the CD38 antibody may be daratumumab or isatuximab.

In some of the methods of the disclosure, daratumumab is administered to the patient at 16 mg/kg every 28 days. In some of the methods, NEOD001 is administered to the patient at 24 mg/kg every 28 days.

In some of the methods of the disclosure, the duration of treatment with the CD38 antibody in combination with an amyloid light chain antibody is effective to reduce the patient's NT-proBNP levels at least to the levels prior to receiving amyloid light chain antibody treatment. In some of the methods, the duration is effective to reduce the patient's NT-proBNP levels below the levels prior to receiving amyloid light chain antibody treatment.

In some of the methods of the disclosure, the patient has received at least one dose, at least two, at least three, at least four, at least 5-12, or more than 12 doses of the CD38 antibody. Also, the duration of the treatment may be at least 3 months, at least 6 months, at least 9 months, at least 12 months, and may include multiple does of the CD38 antibody.

### EXAMPLES

The following examples have been included to illustrate modes disclosed herein. Certain aspects of the following examples are described in terms of techniques and procedures found or contemplated by the present co-inventors to work well in the practice disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1. Phase 3 Clinical Assessment of NEOD001

A Phase 3 global, multi-center, randomized, double-blind, placebo-controlled clinical study of NEOD001 vs. placebo was conducted in newly diagnosed, treatment-naïve patients with AL amyloidosis and cardiac dysfunction, with both arms of the study receiving standard of care (the “VITAL Study”). Patients were randomized on a 1:1 basis to receive 24 mg/kg of NEOD001or placebo via intravenous infusion every 28 days. All patients received bortezomib based chemotherapy concurrently with NEOD001 or placebo. Placebo was administered as a 250 mL bag of normal saline once every 28 days. Additional information regarding the clinical study design is available on https://clinicaltrials.gov.

### Example 2. Evaluation of Patients Receiving NEOD001 and Daratumumab

Nine patients with AL amyloidosis from the VITAL Study who received treatment with the investigational monoclonal antibody NEOD001 also received treatment with daratumumab at 16 mg/kg, with the first dose split over two days. Patients were treated with daratumumab weekly for eight weeks, then every two weeks for four months, then every 28 days. Of these 9 patients, there were 4 men and 5 women at a median age of 68 years old (range, 52-75 years old) and 261 days from diagnosis (range, 51-2037 days). Median NT-proBNP was 3807 pg/ml (1326-13193 pg/ml). Infusions of NEOD001 and daratumumab were separated by 2 days and were well tolerated without any unexpected toxicity. These nine patients with cardiac involvement had not responded to initial therapy with a bortezomib-based regimen. See FIG. 1.

Eighty-eight % of patients achieved >VGPR with daratumumab+NEOD001 in a median of 33 days and cardiac responses were achieved in <90 days. In contrast, patients who were not part of the VITAL Study and were receiving daratumumab alone (n=10) achieved hematologic and cardiac responses at later times (Table 1). In this study, monoclonal antibodies targeting different amyloid light chain and CD38 were safely combined in patients with systemic AL amyloidosis with cardiac involvement. As shown in Table 1, high rates of hematologic and cardiac responses were achieved with the combination of daratumumab and NEOD001, relative to patients receiving daratumumab alone.

TABLE 1
Patient characteristics and results

| | NEOD001 plus | |
|---|---|---|
| | daratumumab | Daratumumab |
| | (n = 9) | (n = 10) |
| Light chain isotype | lambda (λ): 89% | lambda (λ): 70% |
| | kappa (κ): 11% | kappa (κ): 30% |
| Organ involvement | Cardiac: 88% | Cardiac: 70% |
| | Renal: 44% | Renal: 80% |
| No of prior therapies | 1 | 3 |
| Hematologic response | 8/9 (88%) | 8/10 (80%) |
| (>VGPR) | | |
| Median time to best | 33 (range: | 75 (range: |
| hematologic response | 19-161) | 22-242) |
| (days) | | |
| Median NT-proBNP | 3807 (range: | 960 (range: |
| level at baseline (pg/ml) | 1326-13193) | 369-3134) |
| Cardiac response | 7/8 (88%) | 4/6 (67%) |
| Median time to cardiac | 86 | 115 days |
| response (days) | | |
| Reduction in NT- | 74% | 50% |
| proBNP (median) | | |

FIG. 1 shows a representative response as measured by NT-proBNP of patients with advanced worsening AL cardiac involvement despite treatment with NEOD001 and CyBorD (cyclophosphamide, bortezomib, dexamethasone) followed by the addition of daratumumab to the therapy. The dual antibody combination therapy was able to reverse the deterioration of cardiac response.

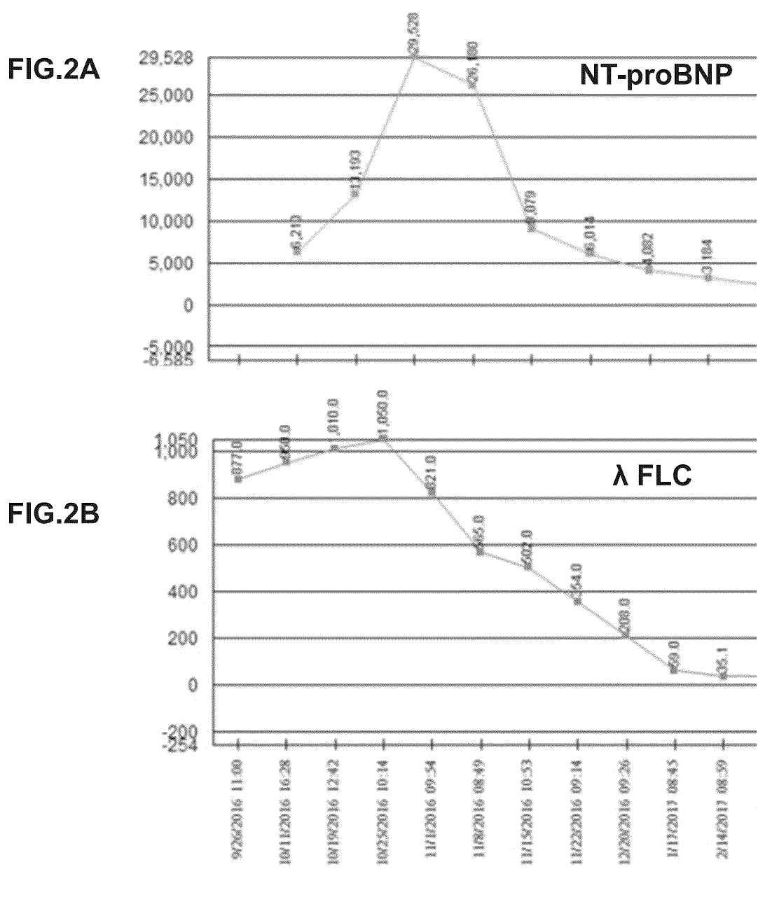

FIG. 2A-B shows the overlap of the two curves showing rapid cardiac improvement based on the NT-proBNP response (FIG. 2A) and gradual lambda light-chain (FIG. 2B) improvement following dual antibody therapy. This pattern is not typical for AL patients experiencing organ responses. Usually the organ response is several months out-of-phase with the light-chain response.

The disclosure of every patent, patent application, and publication cited herein is hereby incorporated herein by reference in its entirety. While this disclosure has been disclosed with reference to specific embodiments, other embodiments and variations of this disclosure can be devised by others skilled in the art without departing from the true spirit and scope of the disclosure. The appended claims include all such embodiments and equivalent variations.

SEQUENCES
Humanized antibody sequence containing Truirine and
human residues (humanized 2A4 light chain
variable region version 3)
SEQ ID NO: 01
DVVMTQSPLSLPVTPGEPASISCRSSQSLVHSTGNTYLHWYLQKPGQSPQLLIYKVSNRFSGVPDRFS
GSGSGTDFTLKISRVEAEDVGVYYCSQSTHVPFTFGGGTKVEIK
Humanized antibody sequence containing murine and
human residues (humanized 2A4 heavy chain
variable region version 3)
SEQ ID NO: 02
EVQLVESGGGLVQPGGSLRLSCAASGFTFNTYAMYWIRQAPGKGLEWVARIRSKSNNYAIYYADSVKD
RFTISRDDSKNSLYLQMNSLKTEDTAVYYCARPYSDSFAYWGQGTLVTVSS
2A4 VL CDR1
SEQ ID NO: 03
RSSQSIVHSTGNTYLH
2A4 VL CDR2
SEQ ID NO: 04
KVSNRFS
2A4 VL CDR3
SEQ ID NO: 05
SQSTHVPFT
2A4 VH CDR1
SEQ ID NO: 06
GFTFNTYAMY
2A4 VH CDR2
SEQ ID NO: 07
RIRSKSNNYAIYYADSVKD
2A4 VH CDR3
SEQ ID NO: 08
PYSDSPAY
7D8 VL CDR1
SEQ ID NO: 09
RSSDSLVHSTGNTYLH
Humanized antibody sequence containing murine and
human residues (humanized. 2A4 kappa light chain
SEQ ID NO: 10
DVVMTQSPLSLPVTDGEPASISCRSSQSLVHSTGNTYLHWYLQKPGOSPQLLIYKVSNRFSGVPDRFS
GSGSGTDFTLKISRVEAEDVGVYYCSQSTHVPFTFGGGTKVEIKRTVAAPSVFIFPPSDEOLKSGTAS
VVCLLNNFYPREAKVQWKVDNALQSGNSQESVTEQDSKDSTYSLSSTLTLSKADYEKEKVYACEVTHQ
GLSSPVTKSFNRGEC
Humanized antibody sequence containing murine and
human residues(humanized 2A4 IgG1 heavy chain
variant. 1 (G1m1 allotype))
SEQ ID NO: 11
EVQDVESGGGLVQPGGSDRLSCAASGFTFNTYAMYWIRQAPGKGLEWVARIRSKSNNYAIYYADSVKD
RFTISRDDSKNSLYLQMNSLKTEDTAVYYCARPYSDSFAYWGQGTLVTVSSASTKGPSVFPLAPSSKS
TSGGTAAIGCLVKDYFPEPVTVSWNSGALTSGVHTFPAVLQSSGLYSLSSVVTVPSSSLGTQTYICNV
NHYPSNTKVDKRVEPKSCDKTHTCPPCPAPELLGGPSVFLFPPKPKDTLMaSRTPEVTCVVVDVSHED
PEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFL
YSKLTVDKSRWOQGNVFSCSVMEEALHNHYTOKSLSLSPGK
Humanized antibody sequence containing murine and
human residues (humanized 2A4 IgG1 heavy chain
variant 2 (G1m3 allotype))
SEQ ID NO: 12
EVQLVESGGGLVQPGGSLRLSCAASGFTFNTYAMYWIRQAPGKGLEWVARIRSKSNNYAIYYADSVKD
RFTISRDDSKNSLYLQMNSLKTEDTAVYYCARDYSDSFAYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
Humanized antibody sequence containing murine and
human residues (humanized 2A4 IgG2 heavy chain)
SEQ ID NO: 13
EVQLVESGGGLVQPGGSLRLSCAASGFTFNTYAMYWIRQAPGKGLEWVARIRSKSNNYAIYYADSVKD
RFTISRDDSKNSLYLQMNSLKTEDTAVYYCARPYSDSFAYWGQGTLVTVSSASTKGPSVFPLAPCSRS
TSESTAALGCLVKDYFPEPVTVSWNSGALTSGVHTFPAVLQSSGLYSLSSVVTVPSSNFGTQTYTCNV
DHKPSNTKVDKTVERKCCVECPPCPAPPVAGPSVFLFPPKPKDTLMISRTPEVTCVVVDVSHEDPEVQ
FNWYVDGVEVHNAKTKPREEQFNSTERVVSVLTVVHQDWLNGKEYKCKVSNKGLPAPIEKTISKTKGQ
PREPQVYTLPPSREEMTKNQVSLTCLVKGFYPSDIAVEWESNGQPENNYKTTPPMLDSDGSFFLYSKL
TVDKSRWQQGNVFSCSVMHEALHNHYTQKSLSLSPGK
SEQ ID NO: 7 from U.S. Pat. No. 7,829,673
SEQ ID NO: 14
QVQLVQSGAEVKKPGSSVKVSCKASGGTFSSYAFSWVRQAPGQGLEWMGRVIPFLGIANSAQKFQGRV
TITADKSTSTAYMDLSSLRSEDTAVYYCARDDIAALGPFDYWGQGTLVTVSSAS
SEQ ID NO: 17 from U.S. Pat. No. 7,829,673
SEQ ID NO: 15
EVQLLESGGGLVQPGGSLRLSCAVSGFTFNSFAMSWVRQAPGKGLEWVSAISGSGGGTYYADSVKGRF
TISRDNSKNTLYLQMNSLRAEDTAVYFCAKDKILWFGEPVEDYWGQGTLVTVSSAS
SEQ ID NO: 27 from U.S. Pat. No. 7,829,673
SEQ ID NO: 16
EVQLVQSGAEVKKPGESLKISCKGSGYSFSNYWIGWVRQMPGKGLEWMGIIYPHDSDARYSPSFQGQV
TFSADKSISTAYLQWSSLKASDTAMYYCARHVGWGSRYWYFDLWGRGTLVTVSS
SEQ ID NO: 2 from U.S. Pat. No. 7,829,673
SEQ ID NO: 17
DIQMTQSPSSLSASVGDRVTITCRASQGISSWLAWYQQKPEKAPKSLIYAASSLQSGVPSRFSGSGSG
TDFTLTISSLQPEDFATYYCQQYNSYPRTFGQGTKVEIK
SEQ ID NO: 12 from U.S. Pat. No. 7,829,673
SEQ ID NO: 18
EIVLTQSPATLSLSPGERATLSCRASQSVSSYLAWYQQKPGQAPRLLIYDASNRATGIPARFSGSGSG
TDFTLTISSLEPEDFAVYYCQQRSNWPPTFGQGTKVEIK
SEQ ID NO: 22 from U.S. Pat. No. 7,829,673
SEQ ID NO: 19
EIVLTQSPATLSLSPGERATLSCRASQSVSSYLAWYQQKPGQAPGLLIYDASNRASGIPARFSGSGSG
TDFTLTISSLEPEDFAVYYCQQRSNWPLTFGGGTKVEIK
SEQ ID NO: 8 from U.S. Pat. No. 7,829,673
SEQ ID NO: 20
SYAFS
SEQ ID NO: 9 from U.S. Pat. No. 7,829,673
SEQ ID NO: 21
RVIPFLGIANSAQKFQ
SEQ ID NO: 10 from U.S. Pat. No. 7,829,673
SEQ ID NO: 22
DDIAALGPFDY
SEQ ID NO: 3 from U.S. Pat. No. 7,829,673
SEQ ID NO: 23
RASQGISSWLA
SEQ ID NO: 4 from U.S. Pat. No. 7,829,673
SEQ ID NO: 24
AASSLQS
SEQ ID NO: 5 from U.S. Pat. No. 7,829,673
SEQ ID NO: 25
QQYNSYPRT
SEQ ID NO: 18 from U.S. Pat. No. 7,829,673
SEQ ID NO: 26
SFAMS
SEQ ID NO: 19 from U.S. Pat. No. 7,829,673
SEQ ID NO: 27
AISGSGGGTYYADSVKG
SEQ ID NO: 20 from U.S. Pat. No. 7,829,673
SEQ ID NO: 28
DKILWFGEPVFDY
SEQ ID NO: 13 from U.S. Pat. No. 7,829,673
SEQ ID NO: 29
RASQSVSSYLA
SEQ ID NO: 14 from U.S. Pat. No. 7,829,673
SEQ ID NO: 30
DASNRAT
SEQ ID NO: 15 from U.S. Pat. No. 7,829,673
SEQ ID NO: 31
QQRSNWPPTF
SEQ ID NO: 5 from U.S. Pat. No. 8,263,746
SEQ ID NO: 32
QVQLVQSGAEVKKPGASVKVSCKASGYTFTSYSINWVRQAPGQGLEWMGYIDPNRGNTNYAQKFQGRV
TMTRDTSISTAYMELSSLRSEDTAVYYCAREYIYFIHGMLDFWGQGTLVTVSS
SEQ ID NO: 13 from U.S. Pat. No. 8,263,746
SEQ ID NO: 33
DIVMTQSPLSLPVTPGEPASISCRSSQSLLFIDGNNYLNWYLQKPGQSPQLLIYLGSNRASGVPDRES
GSGSGTDFTLKISRVEAEDVGVYYCQQYSSKSATFGQGTKVEIKRT
SEQ ID NO: 6 from U.S. Pat. No. 6,263,746
SEQ ID NO: 34
QVQLVESGGGLVQPGGSLRLSCAASGFTFSNYGMHWVRQAPGKGLEWVSNIRSDGSWTYYADSVKGRF
TISRDNSKNTLYLQMNSLRAEDTAVYYCARRYWSKSHASVTDYWGCGTLVTVSS*
SEQ ID NO: 14 from U.S. Pat. No. 8,263,746
SEQ ID NO: 35
DIQMTQSPSSLSASVGDRVTITCRASQDISAFLNWYQQKPGKAPKLLIYKVSNLQSGVPSRFSGSGSG
TDFTLTISSLQPEDFATYYCQQAYSGSITFGQGTKVEIKRT
SEQ ID NO: 7 from U.S. Pat. No. 8,263,746
SEQ ID NO: 36
QVQLVESGGGLVQPGGSLRLSCAASGFTFSSYGMHWVRQAPGKGLEWVSNIYSDGSNTFYADSVKGRF
TISRDNSKNTLYLQMNSLRAEDTAVYYCARNMYRWPFHYFFDYWGQGTLVTVSS
SEQ ID NO: 15 from U.S. Pat. No. 8,263,746
SEQ ID NO: 37
DIELTQPPSVSVAPGQTARISCSGDNIGNKYVSWYQQKPGQAPVVVIYGDNNRPSGIPERFSGSNSGN
TATLTISGTQAEDEADYYCSSYDSSYFVFGGGTKLTVLGQ
SEQ ID NO: 6 from U.S. Pat. No. 6,263,746
SEQ ID NO: 38
QVQLVESGGGLVQPGGSLRLSCAASGFTFSSNGMSWVRQAPGKGLEWVSNISYLSSSTYYADSVKGRF
TISRDNSKNTLYLQMNSLRAEDTAVYYCARFYGYFNYADVWGQGTLVTVSS
SEQ ID NO: 16 from U.S. Pat. No. 8,263,746
SEQ ID NO: 39
DIELTQPPSVSVAPGQTARISCSGDNIGHYYASWYQQKPGQAPVLVIYRDNDRPSGIPERFSGSNSGN
TATLTISGTQAEDEADYYCQSYDYLHDFVFGGGTKLTVLGQ
SEQ ID NO: 22 from U.S. Pat. No. 8,263,746
SEQ ID NO: 40
MANCEFSPVSGDKPCCRLSRRAQLCLGVSILVLILVVVLAVVVPRWRQQWSGPGTTKRFPETVLARCV
KYTEIHPEMRHVDCQSVWDAFKGAFISKHPCNITEEDYQPLMKLGTQTVPCNKILLWSRIKDLAHQFT
QVQRDMFTLEDTLLGYLADDLTWCGEFNTSKINYQSCPDWRKDCSNNPVSVFWKTVSRRFAEAACDVV
HVMLNGSRSKIFDKNSTFGSVEVHNLQPEKVQTLEAWVIHGGREDSRDLCQDPTIKELESIISKRNIQ
FSCKNIYRPDKFLQCVKNPEDSSCTSEI
SEQ ID NO: 41
SKRNIQFSCKNIYR
SEQ ID NO: 42
EKVQTLEAWVIHGG
Heavy chain sequence of DARZALEX ® indicated on
https://www.genome.jp/dbget-bin/www_bget?dr:D10777
SEQ ID NO: 43
EVQLLESGGG LVQPGGSLRL SCAVSGFTFN SFAMSWVRQA PGKGLEWVSA ISGSGGGTYY
ADSVKGRFTI SRDNSKNTLY LQMNSLRAED TAVYFCAKDK ILWFGEPVFD YWGQGTLVTV
SSASTKGPSV FPLAPSSKST SGGTAALGCL VKDYFPEPVT VSWNSGALTS GVHTFPAVLQ
SSGLYSLSSV VTVPSSSLGT QTYICNVNHK PSNTKVDKRV EPKSCDKTHT CPPCPAPELL
GGPSVFLFPP KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
EEMTKNQVSL TCLVKGFYPS DIAVEWESNG QPENNYKTTP PVLDSDGSFF LYSKLTVDKS
RWQQGNVFSC SVMHEALHNH YTQKSLSLSP GK
Light chain sequence of DARZALEX ® indicated on
https://www.genome.jp/dbget-bin/www_bget?dr:D10777
SEQ ID NO: 44
EIVLTQSPAT LSLSPGERAT LSCRASQSVS SYLAWYQQKP GQAPRLLIYD ASNRATGIPA
RFSGSGSGTD FTLTISSLEP EDFAVYYCQQ RSNWPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHY VYACEVTHQG LSSPVTKSFN RGEC
SEQ ID NO: 2 of US 2017/0008966
SEQ ID NO: 45
SKRNIQFSCKNIYR
SEQ ID NO: 3 of US 2017/0008966
SEQ ID NO: 46
EKVQTLEAWVIHGG
SEQ ID NO: 6 of US 2017/0008966
SEQ ID NO: 47
SFAMS
SEQ ID NO: 7 of US 2017/0008966
SEQ ID NO: 48
AISGSGGGTYYADSVK
SEQ ID NO: 8 of US 2017/0008966
SEQ ID NO: 49
DKILWFGEPVFDY
SEQ ID NO: 9 of US 2017/0008966
SEQ ID NO: 50
RASQSVSSYLA
SEQ ID NO: 10 of US 2017/0008966
SEQ ID NO: 51
DASNRAT
SEQ ID NO: 11 of US 2017/0008966
SEQ ID NO: 52
QQRSNWPPTF
SEQ ID NO: 4 of US 2017/0008966
SEQ ID NO: 53
EVQLLESGGGLVQPGGSLRLSCAVSGFTENSFAMSWVRQAPGKGLEWVSAISGSGGGTYYADSVKGRF
TISRDNSKNTLYLQMNSLPAEDTAVYFCAKDKILWFGEPVEDYWGQGTLVTVSS
SEQ ID NO: 5 of US 2017/0008966
SEQ ID NO: 54
EIVLTQSPATLSLSPGERATLSCRASQSVSSYLAWYQQKPGQAPRLLIYDASNRATGIPARFSGSGSG
TDFTLTISSLEPEDFAVYYCQQRSNWPPTFGQGTKVEIK
SEQ ID NO: 12 of US 2017/0008966
SEQ ID NO: 55
EVQLLESGGGLVQPGGSLRLSCAVSGFTFNSFAMSWVRQAPGKGLEWVSAISGSGGGTYYADSVKGRF
TISRDNSKNTLYLQMNSLRAEDTAVYFCAKDKILWFGEPVFDYWGQGTLVTVSSASTKGPSVFPLAPS
SKSTSGGTAALGCLVKDYFPEPVTVSWNSGALTSGVHTFPAVIQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNEALPAPIEKT
ISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 13 of US 2017/0008966
SEQ ID NO: 56
EIVLTQSPATLSLSPGERATLSCRASQSVSSYLAWYQQKPGQAPRLLIYDASNRATGIPARFSGSGSG
TDFTLTISSLEPEDFAVYYCQQRSNWPPTEGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
SEQ ID NO: 14 of US 2017/0008966
SEQ ID NO: 57
QVQLVQSGAEVKKPGSSVKVSCKASGGTFSSYAFSWVRQAPGQGLEWMGRVIPFLGIANSAQKFQGRV
TITADKSTSTAYMDLSSLRSEDTAVYYCARDDIAALGPFDYWGQGTLVTVSSAS
SEQ ID NO: 15 of US 2017/0008966
SEQ ID NO: 58
DIQMTQSPSSLSASVGDRVTITCRASQGISSWLAWYQQKPEKAPKSLIYAASSLQSGVPSRFSGSGSG
TDFTLTISSLQPEDFATYYCQQYNSYPRTFGQGTKVEIK
SEQ ID NO: 16 of US 2017/0008966
SEQ ID NO: 59
EVQLVQSGAEVKKPGESLKISCKGSGYSFSNYWIGWVRQMPGKGLEWMGIIYPHDSDARYSPSFQGQV
TFSADKSISTAYLQWSSLKASDTAMYYCARHVGWGSRYWYFDLWGRGTLVTVSS
SEQ ID NO: 17 of US 2017/0008966
SEQ ID NO: 60
EIVLTQSPATLSLSPGERATLSCRASQSVSSYLAWYQQKPGQAPRLLIYDASNRATGIPARFSGSGSG
TDFTLTISSLEPEDFAVYYCQQRSNWPPTFGQGTKVEIK
SEQ ID NO: 18 of US 2017/0008966
SEQ ID NO: 61
QVQLVESGGGLVQPGGSLRLSCAASGFTFSSYYMNWVRQAPGKGLEWVSGISGDPSNTYYADSVKGRF
TISRDNSKNTLYLQMNSLRAEDTAVYYCARDLPLVYTGFAYWGQGTLVTVSS
SEQ ID NO: 19 of US 2017/0008966
SEQ ID NO: 62
DIELTQPPSVSVAPGQTARISCSGDNLRHYYVYWYQQKPGQAPVLVIYGDSKRPSGIPERFSGSNSGN
TATLTISGTQAEDEADYYCQTYTGGASLVFGGGTKLTVLGQ
SEQ ID NO: 20 of US 2017/0008966
SEQ ID NO: 63
QVQLVQSGAEVAKPGTSVKLSCKASGYTFTDYWMQWVKQRPGQGLEWIGTITPGDGDTGYAQKFQGKA
TLTADKSSKTVYMHLSSLASEDSAVYYCARGDYYGSNSLDYWGQGTSVTVSS
SEQ ID NO: 21 of US 2017/0008966
SEQ ID NO: 64
DIVMTOSELSMSTSLGDPVSITCKASQDVSTVVAWYQQKPGQSPRRLIYSASYRYIGVPDRFTGSGAG
TDFTFTISSVQAEDLAVYYCQQHYSPPYTFGGGTKLEIK

1. A pharmaceutical composition comprising an amyloid light chain antibody and a CD38 antibody.

2. The pharmaceutical composition of claim 1, wherein the amyloid light chain antibody comprises a light chain variable region comprising three complementarity determining regions set forth as SEQ ID NOs: 3, 4, and 5, and a heavy chain variable region comprising three complementarity determining regions set forth as SEQ ID NOs: 6, 7, and 8.

3. The pharmaceutical composition of claim 1, wherein the light chain variable region of the amyloid light chain antibody comprises the amino acid sequence set forth as SEQ ID NO: 1.

4. The pharmaceutical composition of claim 1, wherein the heavy chain variable region of the amyloid light chain antibody comprises the amino acid sequence set forth as SEQ ID NO: 2.

5. The pharmaceutical composition of claim 1, wherein the light chain variable region of the amyloid light chain antibody comprises the amino acid sequence set forth as SEQ ID NO: 1 and the heavy chain variable region of the amyloid light chain antibody comprises the amino acid sequence set forth as SEQ ID NO: 2.

6. The pharmaceutical composition of claim 1, wherein the amyloid light chain antibody comprises a light chain comprising the amino acid sequence set forth as SEQ ID NO: 10 and a heavy chain comprising the amino acid sequence set forth as SEQ ID NO: 11, 12, or 13.

7. The pharmaceutical composition of claim 6, wherein the amyloid light chain antibody comprises a light chain comprising the amino acid sequence set forth as SEQ ID NO: 10 and a heavy chain comprising the amino acid sequence set forth as SEQ ID NO: 12.

8. The pharmaceutical composition of claim 1, wherein the amyloid light chain antibody is birtamimab.

9. The pharmaceutical composition of claim 1, wherein the CD38 antibody comprises a heavy chain variable region comprising the amino acid sequence set forth in SEQ ID NO: 14 or 15.

10. The pharmaceutical composition of claim 1, wherein the CD38 antibody comprises a light chain variable region comprising the amino acid sequence set forth in SEQ ID NO: 17 or 18.

11. The pharmaceutical composition of claim 1, wherein the CD38 antibody comprises heavy and light chain variable region amino acid sequences as set forth in (a) SEQ ID NOs:_14 and 17, respectively; (b) SEQ ID NOs:_15 and 18, respectively; (c) SEQ ID NOs:_16 and 19, respectively; (d) SEQ ID NOs: 43 and 44, respectively; (e) SEQ ID NOs: 53 and 54, respectively; (f) SEQ ID NOs: 57 and 58, respectively; (g) SEQ ID NOs: 59 and 60, respectively; (h) SEQ ID NOs:_61 and 62, respectively; or (i) SEQ ID NOs:_63 and 64, respectively.

12. The pharmaceutical composition of claim 1, wherein the CD38 antibody comprises a heavy chain variable region comprising CDR1, CDR2, and CDR3 sequences comprising the amino acid sequences set forth in SEQ ID NOs: 47, 48, and 49, respectively, and a light chain variable region comprising CDR1, CDR2, and CDR3 sequences comprising the amino acid sequences set forth in SEQ ID NOs: 50, 51, and 52, respectively.

13. The pharmaceutical composition of claim 1, wherein the CD38 antibody comprises a heavy chain variable region comprising CDR1, CDR2, and CDR3 sequences comprising the amino acid sequences set forth in SEQ ID NOs:_20, 21, and 22, respectively, and a light chain variable region comprising CDR1, CDR2, and CDR3 sequences comprising the amino acid sequences set forth in SEQ ID NOs:_23, 24, and 25, respectively.

14. The pharmaceutical composition of claim 1, wherein the CD38 antibody comprises a heavy chain variable region comprising CDR1, CDR2, and CDR3 sequences comprising the amino acid sequences set forth in SEQ ID NOs: 26, 27, and 28, respectively, and a light chain variable region comprising CDR1, CDR2, and CDR3 sequences comprising the amino acid sequences set forth in SEQ ID NOs: 29, 30 and 31, respectively.

15. The pharmaceutical composition of claim 1, wherein the CD38 antibody comprises a heavy chain variable region comprising CDR1, CDR2, and CDR3 sequences from the antibody comprising the amino acid sequence set forth in SEQ ID NO: 32, and a light chain variable region comprising CDR1, CDR2, and CDR3 sequences from the antibody comprising the amino acid sequences set forth in SEQ ID NO: 33.

16. The pharmaceutical composition of claim 1, wherein the CD38 antibody comprises a heavy chain variable region comprising CDR1, CDR2, and CDR3 sequences from the antibody comprising the amino acid sequence set forth in SEQ ID NO: 34, and a light chain variable region comprising CDR1, CDR2, and CDR3 sequences from the antibody comprising the amino acid sequences set forth in SEQ ID NO: 35.

17. The pharmaceutical composition of claim 1, wherein the CD38 antibody comprises a heavy chain variable region comprising CDR1, CDR2, and CDR3 sequences from the antibody comprising the amino acid sequence set forth in SEQ ID NO: 36, and a light chain variable region comprising CDR1, CDR2, and CDR3 sequences from the antibody comprising the amino acid sequences set forth in SEQ ID NO: 37.

18. The pharmaceutical composition of claim 1, wherein the CD38 antibody comprises a heavy chain variable region comprising CDR1, CDR2, and CDR3 sequences from the antibody comprising the amino acid sequence set forth in SEQ ID NO: 38, and a light chain variable region comprising CDR1, CDR2, and CDR3 sequences from the antibody comprising the amino acid sequences set forth in SEQ ID NO: 39.

19. The pharmaceutical composition of claim 1, wherein the CD38 antibody is daratumumab.

20. The pharmaceutical composition of claim 1, wherein the CD38 antibody comprises a heavy chain variable region comprising the amino acid sequence set forth as SEQ ID NO: 43, and a light chain variable region comprising the amino acid sequence set forth as SEQ ID NO: 44.

21. The pharmaceutical composition of claim 1, wherein the CD38 antibody is isatuximab.

22. The pharmaceutical composition of claim 1, wherein the pharmaceutical composition further comprises a buffer.

23. The pharmaceutical composition of claim 22, wherein the buffer is a histidine buffer.
